(19) United States
(12) Patent Application Publication
(10) Pub. No.: US 2017/0226044 A1
(43) Pub. Date: Aug. 10, 2017

(54) PROCESS FOR PRODUCING TRIMETHYLHEXAMETHYLENEDIAMINE

(71) Applicants: Anne Rittsteiger, Olfen (DE); Alexander Martin Ruefer, Recklinghausen (DE); Sven Schneider, DatteIn (DE); Stefan Roeder, Sinntal (DE); Monika Berweiler, Maintal (DE)

(72) Inventors: Anne Rittsteiger, Olfen (DE); Alexander Martin Ruefer, Recklinghausen (DE); Sven Schneider, DatteIn (DE); Stefan Roeder, Sinntal (DE); Monika Berweiler, Maintal (DE)

(73) Assignee: Evonik Degussa GmbH, Essen (DE)

(21) Appl. No.: 15/416,216

(22) Filed: Jan. 26, 2017

(30) Foreign Application Priority Data
Feb. 5, 2016 (EP) 16154364

(51) Int. Cl. C07C 209/48; B01J 35/02; B01J 23/86

(52) U.S. Cl. CPC C07C 209/48; B01J 23/864; B01J 35/023

(57) ABSTRACT

Trimethylhexamethylenediamine is produced by hydrogenating a trimethylhexamethylenedinitrile-comprising mixture in the presence of at least ammonia and hydrogen and a catalyst in the presence or absence of solvent, wherein the catalyst has the following properties: I. after activation the catalyst in its entirety has the following composition in weight percent (wt %), wherein the proportions add up to 100 wt %, based on the metals present: cobalt: 55 to 95 wt %, aluminum: 5 to 45 wt %, chromium: 0 to 3 wt %, and nickel: 0 to 7 wt %, and II. the catalyst is present in the form of irregular particles as granulate and after activation has particle sizes of 1 to 8 mm.

### BACKGROUND OF THE INVENTION

Field of the Invention

The invention relates to an improved process for producing trimethylhexamethylenediamine, hereinbelow referred to as TMD for short, by hydrogenation of trimethylhexamethylenedinitrile, hereinbelow referred to as TMN for short, in the presence of a heterogeneous catalyst.

Discussion of the Background

TMD finds use as an epoxy resin curing agent, as an amine component in polyamides and as a starting component for trimethyhexamethylene diisocyanate which in turn is a starting component for polyurethane systems.

TMD is Produced industrially by hydrogenation of TMN:

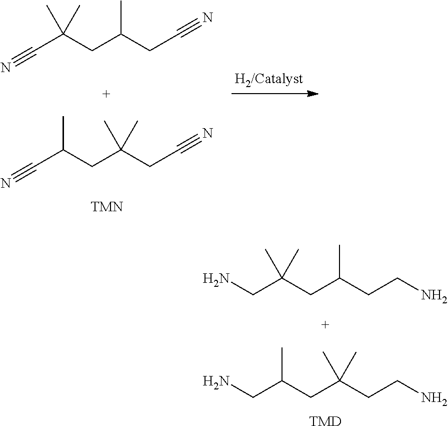

As a consequence of the production process of the TMN a mixture of 2,4,4-trimethylhexamethylenedinitrile and 2,2,4-trimethylhexamethylenedinitrile is employed in the hydrogenation (ratio of about 60:40). Hydrogenation affords a corresponding isomer mixture of 2,4,4-trimethylhexamethylenediamine and 2,2,4-trimethylhexamethylenediamine.

Activated metal catalysts are known as Raney-type catalysts in the chemical industry. They are predominantly employed as powder catalysts in a large number of hydrogenation reactions. Raney-type catalysts are produced from an alloy consisting of at least one catalytically active metal component and at least one leachable alloying component. The catalytically active components employed are mainly nickel, cobalt, copper and iron. The leachable alloying constituent used is predominantly aluminum, though zinc and silicon are also suitable. The so-called Raney alloy is typically finely ground and the leachable component is subsequently completely or partly removed by leaching with alkalis, for example sodium hydroxide solution.

Powder catalysts have the disadvantage that they can only be employed in batch processes. Various processes enabling production of activated metal fixed-bed catalysts have therefore already been described. Such fixed-bed Raney catalysts are particularly suitable for the large industrial scale production of TMD since they allow a process to be run continuously.

DE 43 45 265 and DE 43 35 360 describe shaped Raney catalysts based on Ni, Co, Cu and Fe which are suitable for the hydrogenation of organic compounds. The disadvantage of the catalysts is that metal powder having a lower activity compared to the catalytically active metal needs to be added to the catalysts as a binder.

EP 880 996 describes the production of shaped Raney catalysts which are produced without addition of metallic binders and can be used for the hydrogenation of nitriles. To produce these catalysts a metal-aluminum alloy present as a powder is mixed with a high molecular weight polymer and optionally promoters and then shaped into shaped articles, for example by extrusion. The shaped articles are subsequently calcined at temperatures of up to 850° C. The temperature treatment results in controlled decomposition of the polymer and in the formation of a fixed bed catalyst having sufficient mechanical stability. This is followed by activation by partial or complete leaching of the aluminum using sodium hydroxide solution.

EP 2 114 859 discloses a process for producing TMD from TMN where a shaped article is used as the Raney-type catalyst for the hydrogenation. This catalyst is produced by spray application of a suspension consisting of at least the alloy powder and at least one solvent onto a support material. Binders and promoters may optionally be added to the suspension. The disadvantage is the costly and complex production of the catalyst via a multi-stage process consisting of providing and spray-applying the alloying suspension onto the support material, drying, calcining and activation. A further disadvantage is the mechanical stability/adhesion of the catalytically active layer on the surface of the support material.

### SUMMARY OF THE INVENTION

The present invention has for its object the development of a process for producing TMD from TMN where Raney hydrogenation catalysts which can be produced by a simpler process than fixed bed catalysts are employed and the same or better TMD selectivities are nevertheless achieved than with the hitherto known processes in which Raney hydrogenation catalysts are employed.

It has now been found that, surprisingly, it is possible to achieve a hydrogenation of TMN to TMD over a heterogeneous catalyst with a very high selectivity.

The present invention provides a process for producing trimethylhexamethylenediamine by hydrogenation of trimethylhexamethylenedinitrile-comprising mixtures in the presence of at least ammonia and hydrogen and a catalyst in the presence or absence of solvent,

wherein the catalyst has the following properties:

I.

After activation the catalyst in its entirety has the following composition in weight percent (wt %), wherein the proportions add up to 100 wt %, based on the metals present:

cobalt: 55 to 95 wt %

aluminum: 5 to 45 wt %

chromium: 0 to 3 wt %

nickel: 0 to 7 wt %

and

II.

The catalyst is present in the form of irregular particles as granulate and after activation has particle sizes of 1 to 8 millimetres (mm).

### DETAILED DESCRIPTION OF THE INVENTION

Catalyst

The catalyst consists of a metal alloy, the metal alloy having been surface activated by bases. To this end aluminum is partly or completely leached out of the alloy. The layer thickness of the activated layer on the particle surface of the catalyst is preferably 50 to 1000 micrometres (m). However, it may also be greater or smaller. Accordingly, the catalytically active composition of the catalyst is located on the surface.

After activation the inventive catalyst is present as granulate in the form of individual particles.

After activation the inventive catalyst in its entirety has the following composition in weight percent (wt %), wherein the proportions add up to 100 wt %, based on the metals present:

1st variant

cobalt: 55 to 95 wt %

aluminum: 5 to 45 wt %

chromium: 0 to 3 wt %

nickel: 0 to 7 wt %

or

2nd variant

cobalt: 55 to 90 wt %

aluminum: 5 to 44.5 wt %

chromium: 0.5 to 5 wt %

or

3rd variant

cobalt: 55 to 88 wt %

aluminum: 5 to 44.5 wt %

nickel: 0.5 to 7 wt %

or

4th variant

cobalt: 55 to 85 wt %

aluminum: 5 to 43.5 wt %

chromium: 0.5 to 3 wt %

nickel: 1 to 7 wt %

5th variant

cobalt: 57 to 84 wt %

aluminum: 10 to 40 wt %

chromium: 1 to 2 wt %

nickel: 2 to 4 wt %

“Entirety” is to be understood as meaning that in the composition no distinction is made between the content of the metals on the surface and in the activated layer and in the core of the catalyst particles but rather everything is added and calculated together.

The catalyst is present in the form of irregular particles, i.e. of granulate.

In addition, after activation the inventive catalyst has the following particle sizes:

In general the catalyst, i.e. the granulate particles, may have particle sizes of 1 to 8 millimetres (mm).

In a first preferred variant of the invention the particle sizes of the catalyst, i.e. the granulate particles, vary from 2.5 to 6 millimetres (mm).

In a second preferred variant of the invention the particle sizes of the catalyst, i.e. the granulate particles, vary from 3 to 7 millimetres (mm).

In a third preferred variant of the invention the particle sizes of the catalyst, i.e. the granulate particles, vary from 2 to 5 millimetres (mm).

The particle sizes reported may also have a statistical size distribution within the ranges. Both narrow distributions and broad distributions are in accordance with the invention.

The determination of the particle sizes is described in DIN ISO 9276-1 (September 2004) and 9276-2 (February 2006) and 9276-4 (February 2006) and 9276-6 (January 2012). In addition, exact particulars concerning the definition of particle sizes, the distribution of particle sizes and the measurement of particle sizes may be found in HORIBA® Scientific, A GUIDEBOOK TO PARTICLE SIZE ANALYSIS, 2012, from HORIBA® Instruments, Inc, Irvine, USA.

According to the invention, the distribution of the particle sizes and the measurement of the particle sizes can be determined by laser methods (ISO 13320, 2012), light methods or imaging methods.

The inventive catalyst is preferably obtained by sieving the granulates produced. This produces what are called sieve fractions. Individual sieve fractions may be mixed or a catalyst is obtained by one-off or repeated sieving. The catalysts thus produced have a statistical distribution of particle sizes, typically in the form of a Gaussian distribution. Symmetric and also asymmetric distributions are possible.

In a fourth preferred variant of the invention the particle sizes of the catalyst, i.e. the granulate particles, vary with a statistical distribution between 2.5 to 5.5 millimetres (mm), and wherein up to 10% of the particles may be above the upper limit mentioned and up to 10% of the particles may be below the lower limit mentioned.

In a fifth preferred variant of the invention the particle sizes of the catalyst, i.e. the granulate particles, vary with a statistical distribution between 3.5 to 6.5 millimetres (mm), and wherein up to 10% of the particles may be above the upper limit mentioned and up to 10% of the particles may be below the lower limit mentioned.

In a sixth preferred variant of the invention the particle sizes of the catalyst, i.e. the granulate particles, vary with a statistical distribution between 2 to 5 millimetres (mm), and wherein up to 10% of the particles may be above the upper limit mentioned and up to 10% of the particles may be below the lower limit mentioned.

In a seventh preferred variant of the invention the particle sizes of the catalyst, i.e. the granulate particles, vary with a statistical distribution between 3 to 7 millimetres (mm), and wherein up to 10% of the particles may be above the upper limit mentioned and up to 10% of the particles may be below the lower limit mentioned.

Suitable methods and descriptions of sieve analysis are given in: DIN 66165-1:1987-04 Particle size analysis; sieve analysis; general principles, and in DIN 66165-2:1987-04 Particle size analysis; sieve analysis; procedure.

Paul Schmidt, Rolf Körber, Matthias Coppers: Sieben and Siebmaschinen: Grundlagen and Anwendung. Wiley-VCH Verlag, 2003, ISBN 9783527302079, chapter 4.4: Analysesiebung.

Jörg Hoffmann: Handbuch der Messtechnik. Hanser Verlag, 2007, ISBN 978-3-446-40750-3, chapter 3.12.16.2.1.

It is particularly preferable when after activation the inventive catalyst in its entirety has the following composition in weight percent (wt %), wherein the proportions add up to 100 wt %, based on the metals present:

cobalt: 57 to 84 wt %

aluminum: 10 to 40 wt %

chromium: 1 to 2 wt %

nickel: 2 to 4 wt %

and with

particle sizes of the catalyst, i.e. the granulate particles, having a statistical distribution between 2.5 to 5.5 millimetres (mm),

or

particle sizes of the catalyst, i.e. the granulate particles, having a statistical distribution between 3.5 to 6.5 millimetres (mm),

or

particle sizes of the catalyst, i.e. the granulate particles, having a statistical distribution between 2 to 5 millimetres (mm),

or

particle sizes of the catalyst, i.e. the granulate particles, having a statistical distribution between 3 to 7 millimetres (mm),

and wherein up to 10% of the particles may be above the upper limit mentioned and up to 10% of the particles may be below the lower limit mentioned.

General Method for Production of the Catalyst:

a) Production of the Alloy

The alloy is produced by thermal means, for example in an induction oven. This involves melting the metals to obtain an alloy. For further processing the finished melt is cast into bars for example.

b) Production of the Granulates

The alloy is processed to afford granulates in suitable equipment, for example precomminuted by means of a jaw crusher and subjected to further grinding by means of a roll mill A screening step gives the desired size distribution of the granulates through choice of appropriate sieves (e.g. 3 to 7 mm).

c) Activation of the Catalyst

The catalyst is activated in suitable apparatus. Organic or inorganic bases may be employed. Preference is given to using a lye (e.g. sodium hydroxide solution) where, by an exothermic process, a portion of the aluminum is leached out of the alloy to form hydrogen and aluminate liquor. The concentration of the lye may be between the 5 and 30 wt % and the reaction temperature between 50° C. and 120° C. The degree of activation is determined by the temperature and the reaction time. The required reaction time for a certain degree of activation is directly dependent on the chosen reaction conditions. After activation, the catalyst is washed with water and then stored under water.

Other compositions may be produced analogously in production step a) through appropriate choice of metal amounts.

The catalyst is preferably produced in the sequence described. However, the catalyst may also be activated prior to the production of the granulates.

To increase activity, selectivity and/or service life, the catalysts may additionally comprise doping metals or other modifiers. Typical doping metals are for example Mo, Fe, Ag, V, Ga, In, Bi, Ti, Zr and Mn and also the rare earths alone or in mixtures. Typical modifiers are, for example, those with which the acid-base properties of the catalysts may be influenced, preferably alkali metals and alkaline earth metals or compounds thereof, preferably magnesium and lithium compounds. If such compounds are present, in an amount of not more than about 5 wt %, there is a corresponding reduction in the proportion of the abovementioned metals Co and Al and, if present, Cr and Ni in the catalyst, the proportions of Co and Al and, if present, Cr and Ni after activation adding up to at least 95 wt %, based on the metals present.

The inventive process for producing TMD may be performed batchwise or continuously. The hydrogenation is preferably performed continuously in fixed bed reactors which can be operated in downflow or upflow mode. Suitable reactor types are, for example, shaft furnaces, tray reactors or tube bundle reactors. It is also possible to connect a plurality of fixed-bed reactors in series for the hydrogenation, each of the reactors being operated in downflow mode or in upflow mode as desired.

The hydrogenation is typically effected at temperatures between 20° C. and 150° C., preferably 40° C. and 130° C., and pressures of 0.3 to 50 MPa, preferably 5 to 30 MPa.

Various methods known to one skilled in the art are suitable for controlling the temperature profile in the reactor and in particular for limiting the maximum temperature. Thus, for example, the reactor may be operated entirely without additional reactor cooling, the reaction medium taking up all of the energy released and conveying it out of the reactor by convection. Also suitable are, for example, tray reactors with intermediate cooling, the use of hydrogen circuits with gas cooling, the recycling of a portion of the cooled product (circulation reactor) and the use of external coolant circuits, particularly in the case of tube bundle reactors.

The hydrogen required for the hydrogenation may be supplied to the reactor either in excess, for example at up to 10 000 molar equivalents, or merely in an amount such that the hydrogen consumed by reaction, and the portion of the hydrogen that leaves the reactor dissolved in the product stream, is replenished. In the case of a continuous mode of operation, the hydrogen may be supplied in cocurrent or countercurrent.

In a preferred embodiment the hydrogenation of TMN to TMD over the catalysts to be employed in accordance with the invention is effected in liquid ammonia as solvent. Between 1 and 500 mol, preferably 5 and 200 mol, particularly preferably between 5 and 100 mol, of ammonia are used per mole of TMN.

Although the hydrogenation of TMN to TMD in the presence of ammonia is preferably performed without addition of further solvents, it may also be performed in the presence of additional solvents. Suitable solvents are monohydric alcohols having 1 to 4 carbon atoms, in particular methanol, and ethers, in particular THF, MTBE and dioxane. The substantial advantage of using an additional solvent or solvent mixtures is that the hydrogenation may be performed at lower pressures than when ammonia is employed as the sole solvent.

The required volume of the catalysts to be employed in accordance with the invention is guided by the LHSV value (liquid hourly space velocity) which is dependent on operating pressure, temperature, concentration and catalyst activity and must be adhered to in order to ensure a hydrogenation of the employed TMN that is as complete as possible. When the preferably employed mixture of TMN, ammonia and hydrogen is used the LHSV value is typically between 0.5 and 4 m³ of TMN-ammonia mixture per m³ of catalyst and hour, preferably between 1 and 3 m³/(m³*h)

The reaction mixture leaving the hydrogenation reactor is worked up in a manner known per se. This workup typically comprises removal of the ammonia, of the solvents or mixtures of solvents and ammonia when solvents are present, and isolation of the TMD. The removed ammonia and any further removed solvents are recycled into the process entirely or optionally after discharging of a substream.

The reaction mixture leaving the hydrogenation is further purified by customary methods to obtain TMD of the desired quality. Any standard separation methods, for example distillation, flash evaporation, crystallization, extraction, sorption, permeation, phase separation or combinations of the above, may be employed here. The purification may be performed continuously, batchwise, as a single- or multi-stage procedure, under vacuum or under pressure.

Purification is preferably achieved by distillation under pressure and/or under vacuum in a plurality of steps. Any desired distillation columns with or without internals may be used to this end, for example dephlegmators, dividing walls, unordered internals or random packings, ordered internals or structured packings, or trays with or without forced flow.

In addition to the abovementioned constituents the mixture to be supplied to the hydrogenation reactor may also comprise fractions that are higher- or lower-boiling than TMD and are obtained during the distillative workup of the reaction mixture. Apart from residual TMD, such fractions may also comprise byproducts from which TMD is again formed under reaction conditions. It is particularly advantageous to recycle incompletely converted TMN or aminonitrile-comprising fractions.

It is preferable but not absolutely necessary to initially condition the hydrogenation catalysts to be employed in accordance with the invention with ammonia prior to their use in the hydrogenation. To this end, the catalysts are contacted with ammonia or with mixtures of ammonia and one or more solvents.

Irrespective of whether the process according to the invention is performed in a preferred embodiment or not, one or more hydroxide bases may further be added during the reaction of a mixture of TMN, ammonia, hydrogen and optionally solvent. The addition of hydroxide bases can increase the yield of TMD by reducing byproduct formation. Suitable hydroxide bases are for example alkali metal hydroxides or alkaline earth metal hydroxides. Particularly preferred hydroxide bases are quaternary ammonium hydroxides of general formula (R¹R²R³R⁴N)OH, wherein R¹ to R⁴ may be identical or different and represent aliphatic, cycloaliphatic or aromatic radicals. Examples are tetramethyl-, tetraethyl-, tetra-n-propyl- and tetra-n-butylammonium hydroxide. Suitable concentrations are 0.01 to 100 mmol, preferably 0.05 to 20 mmol, of a tetraalkylammonium hydroxide per mole of TMN.

It is also possible to use one or more cocatalysts in the process according to the invention. Suitable cocatalysts are salts of cobalt, nickel, lanthanum, cerium or yttrium, preferably salts of cobalt and nickel.

The invention also provides for the use of a catalyst for producing trimethylhexamethylenediamine,

wherein the catalyst has the following properties:

I.

After activation the catalyst in its entirety has the following composition in weight percent (wt %), wherein the proportions add up to 100 wt %, based on the metals present:

cobalt: 55 to 95 wt %

aluminum: 5 to 45 wt %

chromium: 0 to 3 wt %

nickel: 0 to 7 wt %

or

cobalt: 55 to 90 wt %

aluminum: 5 to 44.5 wt %

chromium: 0.5 to 5 wt %

or

cobalt: 55 to 88 wt %

aluminum: 5 to 44.5 wt %

nickel: 0.5 to 7 wt %

or

cobalt: 55 to 85 wt %

aluminum: 5 to 43.5 wt %

chromium: 0.5 to 3 wt %

nickel: 1 to 7 wt %

or

cobalt: 57 to 84 wt %

aluminum: 10 to 40 wt %

chromium: 1 to 2 wt %

nickel: 2 to 4 wt %

and

II.

The catalyst is present in the form of irregular particles as granulate and after activation has particle sizes of 1 to 8 millimetres (mm).

Having generally described this invention, a further understanding can be obtained by reference to certain specific examples which are provided herein for purposes of illustration only, and are not intended to be limiting unless otherwise specified.

### Examples

### Example 1: Production of the Inventive Catalyst

a) Production of the Alloy

The alloy was produced in an induction oven. This involved melting the metals in the appropriate amounts at 1500° C. The finished melt was cast into bars for further processing.

b) Production of the Granulates

The alloy bars were precomminuted by means of a jaw crusher and subjected to further grinding by means of a roll mill. The desired size distribution of the granulates was obtained via a sieving step through choice of appropriate screens.

c) Activation of the Catalyst

The catalyst was activated in a standard glass laboratory apparatus, for example a glass beaker. An aqueous lye (e.g. sodium hydroxide solution) was added to the granulates with stirring. The granulates were located in a catalyst basket during activation. The exothermic activation process leached a portion of the aluminum out of the alloy to form hydrogen and sodium aluminate liquor. The employed lye had a concentration of 20 wt % and the reaction temperature was 90° C. The degree of activation was determined via the reaction time. After activation, the catalyst was washed with water and then stored under water.

After activation the employed catalyst in its entirety has the following composition in weight percent (wt %), wherein the proportions add up to 100 wt %, based on the metals present:

cobalt: 75.9 wt %

aluminum: 20.0 wt %

chromium: 1.5 wt %

nickel: 2.6 wt %

A sieve fraction was employed with particle sizes of the catalyst, i.e. of the granulates, having a statistical distribution between 2.0 to 5.0 millimetres (mm), wherein up to 10% of the particles may be above the upper limit mentioned and up to 10% of the particles may be below the lower limit mentioned.

### Example 2: Hydrogenation of TMN

A 2 L batch autoclave having a built-in catalyst basket was used for the hydrogenation of TMN to TMD. Said autoclave was filled with 150 ml of the hydrogenation catalyst to be tested. To condition the catalyst the reactor including the built-in catalyst basket was filled with 1 L of pure ammonia and stirred for 20 hours at about 20° C. After discharging of the solution 700 g of a solution consisting of 15 wt % of IPN in ammonia were added to the reactor and the reaction solution was heated to reaction temperature via an external heating means. The starting point of the hydrogenation was defined by the addition of hydrogen. A pressure of 250 bar was established in the autoclave and the test was run until the respective full conversion based on TMN was achieved.

The composition of the end product was determined by gas chromatography.

The employed reactant TMN had a purity of 96.6%. The TMD selectivity reported in the evaluation was calculated as follows:

$$Selectivity ( TMD ) = \frac{Yield ( TMD )}{Purity ( TMN )} * 100 \%$$

The results of the test series for TMN hydrogenation with the inventive catalyst were summarized in table 1. Full conversion based on TMN was achieved.

TABLE 1
Inventive catalyst:

| Reaction conditions | 80° C., 250 bar | 120° C., 250 bar |
|---|---|---|
| Reaction time | 2 h | 1 h |
| TMD selectivity | 99.6% | 97.7% |

The comparative catalyst employed was a commercial supported cobalt catalyst (tableted shaped article of 3 mm in diameter). The results of the test series for TMN hydrogenation with the comparative catalyst were summarized in table 2. Full conversion based on TMN was achieved.

TABLE 2
Comparative catalyst:

| Reaction conditions | 80° C., 250 bar | 120° C., 250 bar |
|---|---|---|
| Reaction time | 4 h | 2 h |
| TMD selectivity | 99.2% | 94.6% |

The results show a higher activity and selectivity of the inventive catalyst for the hydrogenation of TMN to produce TMD.

European patent application EP16154364 filed Feb. 5, 2016, is incorporated herein by reference.

Numerous modifications and variations on the present invention are possible in light of the above teachings. It is therefore to be understood that within the scope of the appended claims, the invention may be practiced otherwise than as specifically described herein.

1. A process for producing trimethylhexamethylenediamine, comprising:
hydrogenating a trimethylhexamethylenedinitrile-comprising mixture in the presence of at least ammonia and hydrogen and a catalyst in the presence or absence of solvent,
wherein the catalyst has the following properties:
I. after activation the catalyst in its entirety has the following composition in weight percent (wt %), wherein the proportions add up to 100 wt %, based on the metals present:
cobalt: 55 to 95 wt %,
aluminum: 5 to 45 wt %,
chromium: 0 to 3 wt %, and
nickel: 0 to 7 wt %, and
II. the catalyst is present in the form of irregular particles as granulate and after activation has particle sizes of 1 to 8 mm.

2. The process according to claim 1, wherein
I. after activation the catalyst in its entirety has the following composition in weight percent (wt %), wherein the proportions add up to 100 wt %, based on the metals present:
cobalt: 55 to 90 wt %,
aluminum: 5 to 44.5 wt %, and
chromium: 0.5 to 5 wt %.

3. The process according to claim 1, wherein
I. after activation the catalyst in its entirety has the following composition in weight percent (wt %), wherein the proportions add up to 100 wt %, based on the metals present:
cobalt: 55 to 88 wt %,
aluminum: 5 to 44.5 wt %, and
nickel: 0.5 to 7 wt %.

4. The process according to claim 1, wherein
I. after activation the catalyst in its entirety has the following composition in weight percent (wt %), wherein the proportions add up to 100 wt %, based on the metals present:
cobalt: 55 to 85 wt %,
aluminum: 5 to 43.5 wt %,
chromium: 0.5 to 3 wt %, and
nickel: 1 to 7 wt %.

5. The process according to claim 1, wherein
I. after activation the catalyst in its entirety has the following composition in weight percent (wt %), wherein the proportions add up to 100 wt %, based on the metals present:
cobalt: 57 to 84 wt %,
aluminum: 10 to 40 wt %,
chromium: 1 to 2 wt %, and
nickel: 2 to 4 wt %.

6. The process according to claim 1, wherein the particle size of the catalyst is from 2.5 to 6 mm,
or
the particle size of the catalyst is from 3 to 7 mm,
or
the particle size of the catalyst is from 2 to 5 mm.

7. The process according to claim 1, wherein the catalyst comprises granulates and is obtained by sieving the granulates produced.

8. The process according to claim 7, wherein the catalyst comprises granulates and is obtained by sieving the granulates produced, and
the particle size of the catalyst has a statistical distribution between 2.5 to 5.5 mm, or
the particle size of the catalyst has a statistical distribution between 3.5 to 6.5 mm, or
the particle size of the catalyst has a statistical distribution between 2 to 5 mm, or
the particle size of the catalyst has a statistical distribution between 3 to 7 mm, and
wherein up to 10% of the particles may be above the upper limit of the statistical distribution and up to 10% of the particles may be below the lower limit of the statistical distribution.

9. The process according to claim 1, wherein, after activation, the catalyst in its entirety has the following composition in weight percent (wt %), wherein the proportions add up to 100 wt %, based on the metals present:
cobalt: 57 to 84 wt %,
aluminum: 10 to 40 wt %,
chromium: 1 to 2 wt %, and
nickel: 2 to 4 wt %, and
wherein the particle size of the catalyst has a statistical distribution between 2.5 to 5.5 mm,
or
the particle size of the catalyst has a statistical distribution between 3.5 to 6.5 mm,
or
the particle size of the catalyst has a statistical distribution between 2 to 5 mm,
or
the particle size of the catalyst has a statistical distribution between 3 to 7 mm,
wherein up to 10% of the particles may be above the upper limit of the statistical distribution and up to 10% of the particles may be below the lower limit of the statistical distribution.

10. The process according to claim 1, wherein the catalyst further comprises a doping metal.

11. The process according to claim 1, wherein catalyst comprises a modifier.

12. The process according to claim 1, wherein said process is a batchwise or continuous, single-stage or multi-stage process.

13. The process according to claim 1, wherein the hydrogenation is performed continuously in a fixed bed reactor which is operated in downflow or upflow mode.

14. The process according to claim 1, wherein the hydrogenation is performed at a temperature between 20° C. and 150° C., and a pressure of 0.3 to 50 MPa.

15. The process according to claim 10, wherein the doping metal is selected from the group consisting of Mo, Fe, Ag, V, Ga, In, Bi, Ti, Zr, Mn, a rare earth metal and mixtures thereof.

16. The process according to claim 15, wherein the modifier is an alkali metal, and alkaline earth metal or a compound thereof.

17. The process according to claim 16, wherein the modifier is a magnesium and/or lithium compound.

18. The process according to claim 1, wherein the hydrogenation is performed at a temperature between 40° C. and 130° C., and a pressure of 5 to 30 MPa.
